(19) United States
(12) Patent Application Publication
(10) Pub. No.: US 2024/0074938 A1
(43) Pub. Date: Mar. 7, 2024

(54) MODULAR ULTRASONIC MASSAGE RING DEVICE

(71) Applicant: Zhijun PENG, Foshan (CN)

(72) Inventor: Zhijun PENG, Foshan (CN)

(21) Appl. No.: 18/241,912

(22) Filed: Sep. 4, 2023

(30) Foreign Application Priority Data
Sep. 5, 2022 (CN) 202211076895.8

(51) Int. Cl. A61H 23/00; A61H 23/02; A61M 37/00

(52) U.S. Cl. CPC A61H 23/008; A61H 23/02; A61M 37/0092; A61H 2201/1207

(57) ABSTRACT

A modular ultrasonic massage ring device, including arc frame components, assembly locking structures, and ultrasonic massage mechanisms extending radially from the arc frame components respectively; each of the assembly locking structures is arranged between opposing ends of two adjacent arc frame components to assemble and fix the arc frame components to form a ring-shaped ultrasonic massage device; the ultrasonic massage mechanisms extend radially from the arc frame components. A number of the arc frame components to be assembled can be increased or reduced to assemble different massage ring device of different specifications so that a single product can meet the demands of all family members who may have different body shape.

### BACKGROUND OF THE INVENTION

The present invention relates to the field of human body massage/healthcare devices, and in particular, to an ultrasonic massager for human body massage/healthcare.

At present, commercially available ultrasonic massage devices using high-frequency ultrasonic motors generally have fixed specifications and sizes, for example, as disclosed in CN215131668U (Application No. CN202121717898.6) titled “MASSAGING AND UTERUS-WARMING ABDOMINAL BELT WITH GOOD HEAT PRESERVATION PERFORMANCE FOR GYNECOLOGICAL USE”. When more than one family member needs to use the massage device, and due to the differences in body shape of different family members, one massage device with a fixed specification is obviously impossible to meet the use requirements of all the family members. As such, it is necessary to purchase one massage device for each member. However, a massage device is a healthcare device used only occasionally rather than a daily necessity for a family. Accordingly, such massage devices with improper structural design impose a large unnecessary expenditure on the family. Therefore, existing ultrasonic massage devices with fixed specifications and sizes are obviously improper in structural design and cannot meet the real demands of people.

### BRIEF SUMMARY OF THE INVENTION

In view of the aforesaid disadvantages in the prior art, the present invention provides a modular ultrasonic massage ring device, comprising a plurality of arc frame components joined and fixed by assembly locking structures; and an ultrasonic massage mechanism is positioned to extend radially on each of the arc frame components. Therefore, the present invention can flexibly increase or reduce a number of the arc frame components being used according to actual needs so that different ultrasonic massage ring device of different specifications suitable for different family members may be constructed. As such, a single product is enough to satisfy the needs of all family members who may have different body shapes. The present invention has a simple structure, and is simple and convenient to assemble,

The present invention is achieved as follows:

A modular ultrasonic massage ring device, comprising a plurality of arc frame components, a plurality of assembly locking structures, and a plurality of ultrasonic massage mechanisms extending radially from the arc frame components respectively, wherein each of the assembly locking structures is arranged between opposing ends of two adjacent arc frame components, and the arc frame components are assembled with one another through the assembly locking structures to form a ring-shaped ultrasonic massage device.

Further, each of the assembly locking structures comprises a male mating part, a female mating part, a pin, a protrusion part, a protrusion-receiving hole, and pin holes, wherein one end of each of the arc frame components is provided with the male mating part and the protrusion-receiving hole of one of the assembly locking structures, and another end of each of the arc frame components is provided with the female mating part and the protrusion part of another one of the assembly locking structures; the pin holes are vertically arranged through the male mating part and the female mating part respectively, the male mating part and the female mating part are mated together; the protrusion part is inserted into the protrusion-receiving hole, and the pin is inserted through the pin holes, and therefore the male mating part and the female mating part are locked together.

Still further, a through hole is provided on each of the arc frame components extending along a radial direction of the modular ultrasonic massage ring device, a vertically arranged threaded bore is further provided on an outer wall of the through hole, and a threaded locking bolt is installed in the threaded bore.

Still further, each of the ultrasonic massage mechanisms is provided with a rod; the rod of each of the ultrasonic massage mechanisms is accommodated in the through hole of a corresponding arc frame component, and the threaded locking bolt is screwed downwards to abut and lock a position of the rod.

The present invention has the following beneficial effects: The present invention adopts a plurality of arc frame components, and utilizes assembly locking structures to achieve the assembly and fixation of the arc frame components. An ultrasonic massage mechanism is arranged to extend radially from each of the arc frame components, such that, by flexibly increasing or reducing a number of the arc frame components being used for assembly according to actual needs, different massage rings of different sizes and specifications may be assembled to satisfy the demands for all family members who may have different body shapes. The product features a simple structure, ease and convenience of assembly, capability of disassembling for packaging, small size, and portability.

### BRIEF DESCRIPTION OF THE DRAWINGS

FIG. 1 is a structural schematic view of the present invention.

FIG. 2 is a structural schematic view of the present invention in a partially disassembled state.

FIG. 3 is a first structural schematic view of an arc frame component and a corresponding ultrasonic massage mechanism of the present invention in a disassembled state.

FIG. 4 is a partial cross-sectional view of the ultrasonic massage mechanism of the present invention in a disassembled state.

FIG. 5 is a second structural schematic view of the arc frame component and another kind of ultrasonic massage mechanism of the present invention in a disassembled state.

FIG. 6 is a third structural schematic of the arc frame component and yet another kind of ultrasonic massage mechanism of the present invention in a disassembled state

### DETAILED DESCRIPTION OF THE INVENTION

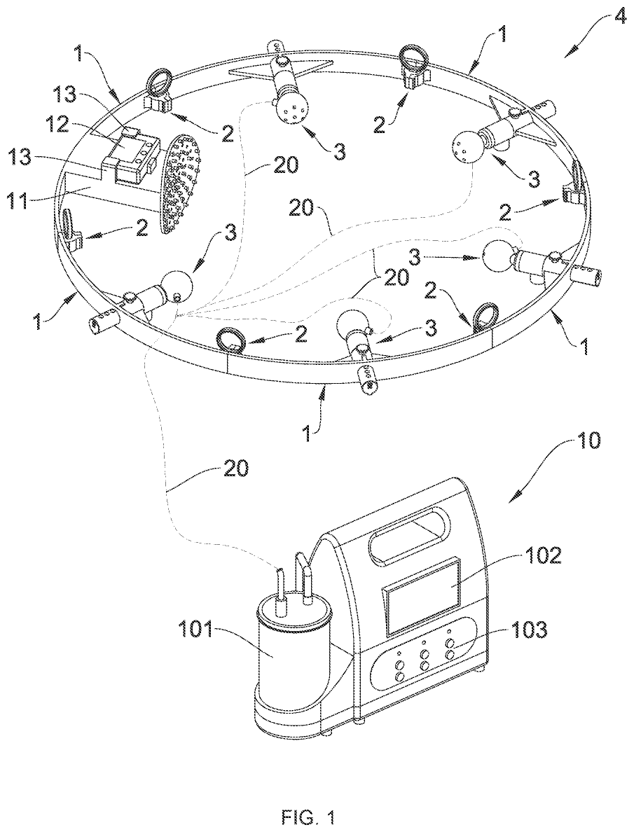

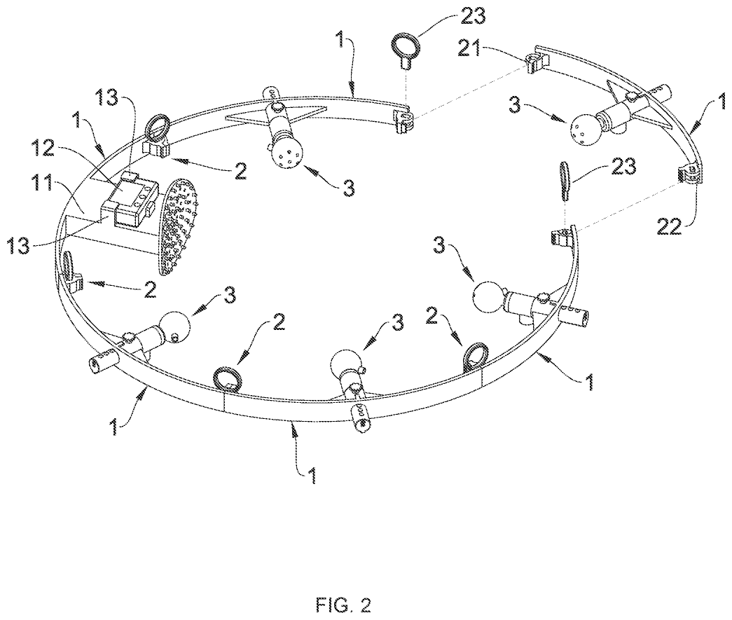

As shown in FIGS. 1 and 2, the present invention provides a modular ultrasonic massage ring device, comprising a plurality of arc frame components 1, a plurality of assembly locking structures 2, and a plurality of ultrasonic massage mechanisms 3 extending radially from the arc frame components 1 respectively, wherein each of the assembly locking structures 2 is arranged between opposing ends of two adjacent arc frame components 1, and the arc frame components 1 are assembled with one another through the assembly locking structures 2 to form a ring-shaped ultrasonic massage device 4. Through such a structural design, a number of the arc frame components to be assembled can be flexibly increased or reduced as desired, so that massage rings of different sizes and specifications may be assembled such that a single product is enough to satisfy the needs of all family members who may have different body shapes. The arc frame components 1 and the assembly locking structures 2 are generally made of a plastic material or a nylon material, which imparts a certain elasticity to prevent breakage of the arc frame components and the assembly locking structures.

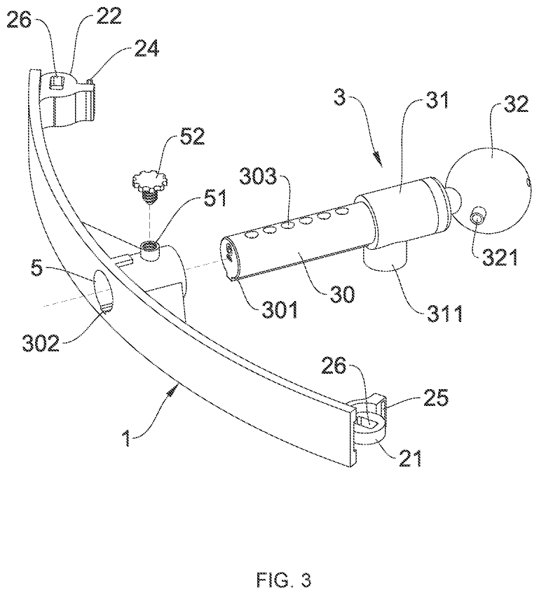

As shown in FIG. 2 and FIG. 3, in order that the assembly locking structures 2 are structurally simple and easy to use, each of the assembly locking structure 2 comprises a male mating part 21, a female mating part 22, a pin 23, a protrusion part 24, a protrusion-receiving hole 25, and pin holes 26, wherein one end of each of the arc frame components is provided with the male mating part 21 and the protrusion-receiving hole 25 of one of the assembly locking structures 2, and another end of each of the arc frame components 1 is provided with the female mating part 22 and the protrusion part 24 of another one of the assembly locking structures 2; the pin holes 26 are vertically arranged through the male mating part 21 and the female mating part 22 respectively, the male mating part 21 and the female mating part 22 are mated together; the protrusion part 24 is inserted into the protrusion-receiving hole, and the pin 23 is inserted through the pin holes 26, and therefore the male mating part 21 and the female mating part 22 are locked together, as shown in FIG. 1.

As shown in FIG. 3, in order to enable adjustment of the ultrasonic massage mechanisms 3 along a radial direction of the modular ultrasonic massage ring device so that massage heads of the ultrasonic massage mechanisms can be adjusted of their pressure against human body so as to obtain different massage forces, a through hole 5 is provided on each of the arc frame components 1 extending along a radial direction of the modular ultrasonic massage ring device, a vertically arranged threaded bore 51 is further provided on an outer wall of the through hole 5, and a threaded locking bolt 52 is installed in the threaded bore 51. Correspondingly, each of the ultrasonic massage mechanisms 3 is provided with a rod 30; a plurality of adjusting recesses 303 are arranged on the rod 30; the rod 30 of each of the ultrasonic massage mechanisms 3 is accommodated in the through hole 5 of a corresponding arc frame component 1, and the threaded locking bolt 52 is screwed downwards and abuts against one of the adjusting recesses 303, by inserting the rod such that the threaded locking bolt 52 can abut against different adjusting recesses 303 as the rod is inserted by different depths, the rod 30 is movable along a radial direction of the modular ultrasonic massage ring device in the through hole 5 and being fixed in position by the threaded locking bolt. In use, a user may first unscrew the threaded locking bolt 52 to pull the corresponding ultrasonic massage mechanism 3 so as to adjust the ultrasonic massage mechanism 3 to a proper pressure against the human body, and then screw the threaded locking bolt 52 down again to complete the adjustment process. In order to prevent the rod 30 from rotating in a circumferential direction of the through hole 5 and to enable a tighter locking via the threaded locking bolt 52, as shown in FIG. 3, a guide protrusion 301 and a guide groove 302 are further arranged on the rod 30 of each of the ultrasonic massage mechanisms 3 and in the through hole 5 of the corresponding arc frame component 1 respectively. The guide protrusion 301 and the guide groove 302 achieve a guided limitation, which can effectively prevent the rod 30 from rotating in the circumferential direction of the through hole 5, and enable a tighter locking via the threaded locking bolt 52 by enabling easier and more accurate insertion of the threaded locking bolt 52 into the adjusting recesses 303.

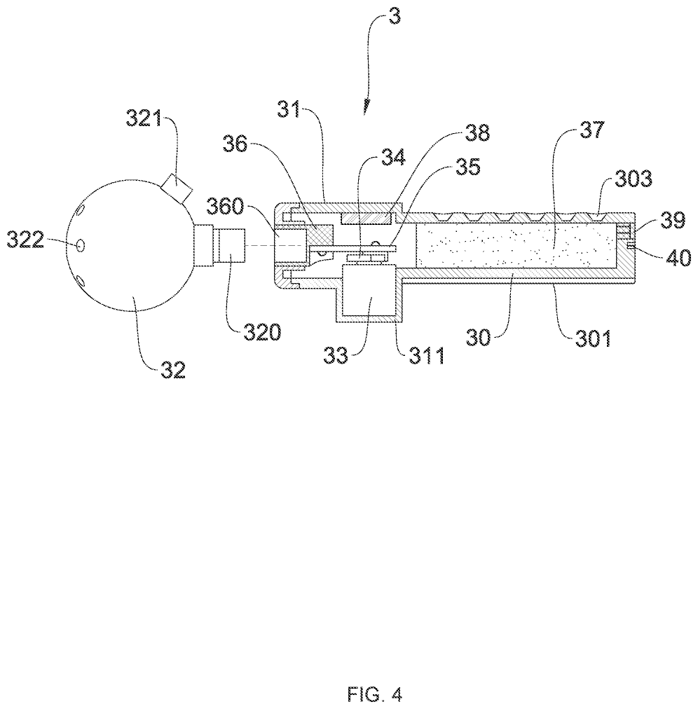

As shown in FIG. 4, each of the ultrasonic massage mechanisms 3 comprises a housing 31, a massage head 32, a motor 33, an eccentric wheel 34, a reciprocating rod 35, and a connector 36. The motor 33 is installed in a motor base 311 arranged in the housing 31. The eccentric wheel 34 is installed on a rotating shaft of the motor 33. The connector 36 is movably received in a hole arranged at a front end of the housing 31. One end of the reciprocating rod 35 is connected with the eccentric wheel 34, while another end of the reciprocating rod 35 is connected with the connector 36. The massage head 32 is connected to the connector 36. The massage head 32 is provided with a connecting protrusion 320, the connector 36 is provided with a receiving cavity 360, and the connecting protrusion 320 is accommodated in the receiving cavity 360 to achieve connection. In operation, the motor 33 drives the eccentric wheel 34 to actuate the reciprocating rod 35 to move back and forth at a high speed. The massage head 32 is thus driven to hit the muscles of a human body at a high speed, thus effectively relaxing the muscles, promoting cell activity and blood circulation, and regulating metabolism.

In order to make the present invention portable and convenient to use, without constraints of a power supply wire, as shown in FIG. 4, the rod 30 is arranged at a rear end of the housing 31, and a battery 37 is further arranged inside the rod 30. A charging interface 39 and a switch button 40 are further arranged on a rear end surface of the rod 30. The charging interface 39 is configured for charging the storage battery 37, and the switch button 40 is configured for turning the motor 33 on or off. As shown in FIG. 4, a circuit board 38 is further arranged in the housing 31, and the circuit board 38 is configured for controlling a rotation speed of the motor, controlling charge and discharge of the battery 37, and conducting Bluetooth® communication.

As shown in FIGS. 3 and 4, a pipe connector 321 is arranged proximal to a rear end of the massage head 32, and a plurality of uniformly distributed spraying holes 322 are provided at a front end of the massage head 32. As shown in FIG. 1, when the present invention is implemented, an electric pump 10 used for supplying medicine can be provided. The electric pump 10 is connected with the pipe connector 321 through a hose 20. Traditional Chinese medicine in liquid form is pressurized and pumped to the massage heads 32 from a liquid storage tank 101 of the electric pump 10, and is then sprayed on the skin from spraying holes 322 arranged on each of the massage heads 32. The synchronous conduction of vibration massage together with spraying of the liquid medicine can accelerate the absorption of liquid medicine by the skin and thus improve the effects of health care and therapeutic effects. The electric pump 10 is provided with a display 102 and control buttons 103 to facilitate the use of the device by the user.

In addition, in order to facilitate centralized control of the ultrasonic massage mechanisms 3 by the user, as shown in FIG. 1, a platform 11 is further arranged on one of the arc frame components 1. A wireless controller 12 is embedded in the platform 11. The wireless controller 12 establishes a communicative connection with each of the ultrasonic massage mechanisms 3 by using Bluetooth® wireless communication. A control circuit board, a battery, a charging interface, a display, and control buttons are arranged on the wireless controller 12. The wireless controller 12 enables centralized and easy management and control of operations such as starting, speed adjustment, timing, closing, and the like of each of the ultrasonic massage mechanisms 3. The wireless controller 12 can also establish a communicative connection with a smart phone by Bluetooth® wireless communication. A designated application software may be installed on the smart phone for controlling operation of the device of the present invention by using the smart phone. The platform 11 is also provided with a clamping part 13, such that the wireless controller 12 can be conveniently and detachably mounted in a cavity formed in the clamping part 13.

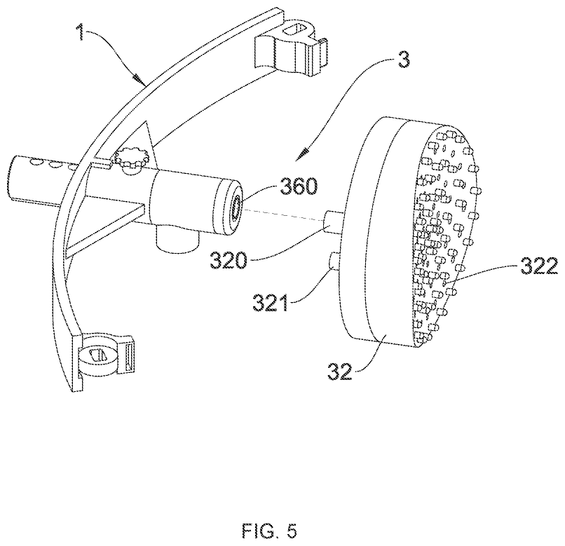

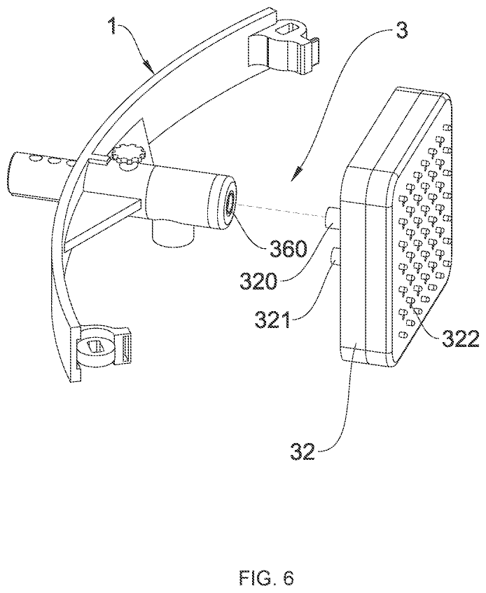

As shown in FIGS. 4, 5, and 6, each of the massage heads 32 can be formed into various shapes, such as a spherical shape, an oblate shape, a rectangular shape, etc., according to the actual needs.

What is claimed is:

1. A modular ultrasonic massage ring device, comprising a plurality of arc frame components, a plurality of assembly locking structures, and a plurality of ultrasonic massage mechanisms extending radially from the arc frame components respectively, wherein each of the assembly locking structures is arranged between opposing ends of two adjacent arc frame components, and the arc frame components are assembled with one another through the assembly locking structures to form a ring-shaped ultrasonic massage device.

2. The modular ultrasonic massage ring device of claim 1, wherein each of the assembly locking structures comprises a male mating part, a female mating part, a pin, a protrusion part, a protrusion-receiving hole, and pin holes, wherein one end of each of the arc frame components is provided with the male mating part and the protrusion-receiving hole of one of the assembly locking structures, and another end of each of the arc frame components is provided with the female mating part and the protrusion part of another one of the assembly locking structures; the pin holes are vertically arranged through the male mating part and the female mating part respectively, the male mating part and the female mating part are mated together; the protrusion part is inserted into the protrusion-receiving hole, and the pin is inserted through the pin holes, and therefore the male mating part and the female mating part are locked together.

3. The modular ultrasonic massage ring device of claim 1, wherein a through hole is provided on each of the arc frame components extending along a radial direction of the modular ultrasonic massage ring device, a vertically arranged threaded bore is further provided on an outer wall of the through hole, and a threaded locking bolt is installed in the threaded bore.

4. The modular ultrasonic massage ring device of claim 3, wherein each of the ultrasonic massage mechanisms is provided with a rod; a plurality of adjusting recesses are arranged on the rod; the rod of each of the ultrasonic massage mechanisms is accommodated in the through hole of a corresponding arc frame component, and the threaded locking bolt is screwed downwards and abuts against one of the adjusting recesses so that the rod is movable in the through hole along the radial direction of the modular ultrasonic massage ring device and capable of being fixed in position by the threaded locking bolt.

5. The modular ultrasonic massage ring device of claim 4, wherein a guide protrusion and a guide groove are further arranged on the rod of each of the ultrasonic massage mechanisms and in the through hole of the corresponding arc frame component respectively.

6. The modular ultrasonic massage ring device of claim 4, wherein each of the ultrasonic massage mechanisms comprises a housing, a massage head, a motor, an eccentric wheel, a reciprocating rod, and a connector; the motor is installed in a motor base arranged in the housing; the eccentric wheel is installed on a rotating shaft of the motor; the connector is movably received in a hole arranged at a front end of the housing; one end of the reciprocating rod is connected with the eccentric wheel, while another end of the reciprocating rod is connected with the connector; the massage head is connected to the connector.

7. The modular ultrasonic massage ring device of claim 6, wherein the rod is arranged at a rear end of the housing, and a battery is further arranged inside the rod.

8. The modular ultrasonic massage ring device of claim 7, wherein a charging interface and a switch button are further arranged on a rear end surface of the rod.

9. The modular ultrasonic massage ring device of claim 6, wherein a circuit board is further arranged in the housing.

10. The modular ultrasonic massage ring device of claim 6, wherein a pipe connector is arranged proximal to a rear end of the massage head, and a plurality of uniformly distributed spraying holes are provided at a front end of the massage head.
